(19) United States
(12) Patent Application Publication
(10) Pub. No.: US 2020/0109491 A1
(43) Pub. Date: Apr. 9, 2020

(54) NANOFIBERS COMPRISING FIBROIN AS WELL AS SYSTEM COMPRISING HYDROGEL AND SAID NANOFIBERS

(71) Applicant: Silk Biomaterials S.R.L., Lomazzo, Como (IT)

(72) Inventors: Vita Guarino, Campi Salentina, Lecce (IT); Ada Potenza, Mola Di Bari, Bari (IT); Valeria Rizzello, Lecce (IT)

(21) Appl. No.: 16/500,932

(22) PCT Filed: Apr. 4, 2018
(86) PCT No.: PCT/IB2018/052317

§ 371 Date: Oct. 4, 2019

(30) Foreign Application Priority Data
Apr. 4, 2017 (IT) 102017000036930

(51) Int. Cl. D01D 5/00; D01F 1/10; A61K 47/36; A61K 9/00; D01F 8/10; D01F 4/02

(52) U.S. Cl. CPC D01D 5/003; D01F 1/10; A61K 47/36; A61K 45/06; D01F 8/10; D01D 5/0076; D01F 4/02; A61K 9/0092

(57) ABSTRACT

Described herein are nanofibers including an outer membrane and a core, wherein the outer membrane is made of fibroin and the core is a biocompatible and biodegradable polymer. Also described herein are a method for obtaining the nanofibers and the use thereof to convey bioactive molecules and/or particles and/or cells and/or in the treatment of diseases. Also described herein are powdered nanofibers, optionally suspended in an aqueous solution, a hydrogel system including the powdered nanofibers, and the use of the powdered nanofibers and of the hydrogel system to convey bioactive molecules and/or particles and/or cells and/or in the treatment of diseases.

The present invention relates to nanofibers (1) comprising an outer membrane (2) and a core (3) wherein said outer membrane (2) is made of fibroin and said core (3) is made of a biocompatible and biodegradable polymer. The present invention further relates to a method for obtaining said nanofibers and to the use thereof to convey bioactive molecules and/or particles and/or cells and/or in the treatment of pathologies. The present invention also relates to said powdered nanofibers, optionally suspended in an aqueous solution, and to a hydrogel system comprising said powdered nanofibers and to the use of said powdered nanofibers and of said system for conveying bioactive molecules and/or particles and/or cells and/or in the treatment of diseases.

### BACKGROUND ART

Silk fibroin is a natural protein fiber produced by various insects (order Lepidoptera), spiders (order Araneae), as well as by various Hymenoptera, dipterans, coleopterans, etc., typically by silkworms of the Bombyx mori species. Silk is of considerable interest as a carrier for drugs, due to its biocompatibility, the possibility of programming its degradation, and its exceptional ability to maintain the functionality of the active ingredients loaded on it.

Electrospinning is a versatile method that allows to obtain nanometric fibers starting from polymeric solutions. This method was initially described in U.S. Pat. No. 1,975,504.

Alessandrino et al. in Eng. Life Sci. 2008, 8, No. 3, 219-225 describe nanofibers obtained from silk fibroin by electrospinning. The electrospinning was performed on silk fibroin using solvents such as hexafluoro-2-propanol (HFIP); hexafluoroacetone (HFA), and formic acid, solvents toxic for humans and the environment (Zarkoob S et al. Structure and morphology of electrospun silk nanofibers. Polymer 2004; 45:3973-3977. Kawahara Y et al. Structure for electro-spun silk fibroin nanofibers. J Appl Polym Sci 2008; 107:3681-3684. Ohgo K et al. Preparation of non-woven nanofibers of Bombyx mori silk. Samia cynthia ricini silk and recombinant hybrid silk with electrospinning method. Polymer 2003; 44:841-846).

WO2009042829 describes nanofibers dispersed in a hydrogel, where the length of said nanofibers is of the order of nanometers or millimeters and they consist of a cross-linked carboxy-functionalized polymer and a hydroxy-functionalized polymer, such as polyacrylic acid (PAA) and a polysaccharide, such as dextran.

Nanofibers obtained from silk fibroin were dispersed in a hydrogel, for example in Elia R et al. J Biomater Appl 2013 27: 749. The used polymer is typically hyaluronic acid.

Synthetic polymers, such as polyethylene oxide (PEO), have been used to obtain nanofibers in combination with chitosan (Pakravan M et al., Biomacromolecules 2012, 13, 412-421).

The need to have a carrier for molecules of biocompatible pharmacological interest, easy to be obtained and injectable, is strongly felt.

### DESCRIPTION OF THE INVENTION

The present invention first relates to a nanofiber (1) comprising an outer membrane (2) and a core (3), wherein said outer membrane (2) is made of fibroin and said core (3) is made of a biodegradable and biocompatible polymer. Preferably, said polymer is water-soluble.

In a preferred embodiment, said nanofibers also comprise, in said core, one or more bioactive molecules and/or particles and/or cells.

A second aspect of the present invention is a method for obtaining said nanofibers, diagrammatically shown in FIG. 8.

In a further aspect, said powdered nanofibers (1) are described and claimed, where powdered nanofibers mean nanofibers having a particle size from 1 to 5000 microns, preferably from 10 to 1000 microns.

In a further aspect, a hydrogel system comprising said nanofibers (1), wherein said nanofibers are present in a powdered form in said hydrogel is described and claimed. In a preferred embodiment, said system also comprises one or more bioactive molecules and/or particles and/or cells. Said bioactive molecules and/or particles and/or cells are in the core of said nanofibers or in the core of said nanofibers and in the hydrogel itself, or in the hydrogel.

The present invention further relates to a method for obtaining said system.

Said nanofibers, or said powdered nanofibers, or said hydrogel system comprising said powdered nanofibers, said nanofibers and/or said hydrogel optionally loaded with one or more bioactive molecules and/or particles and/or cells for use in the treatment of diseases are also claimed. Said nanofibers, or said powdered nanofibers, or said system are administered and positioned in the areas to be treated, allowing a controlled release of the bioactive molecules and/or particles and/or cells optionally contained therein and undergoing to re-uptake, consisting of a biocompatible and biodegradable material.

### DESCRIPTION OF THE DRAWINGS

FIGS. 1-6: picture of the nanofibers according to the present invention, obtained according to Example 1.

FIG. 7: scheme of a nanofiber according to the present invention.

FIG. 8: diagram of the process to obtain nanofibers according to the present invention.

FIG. 9: diagram of the process to obtain the hydrogel+nanofibers system according to the present invention.

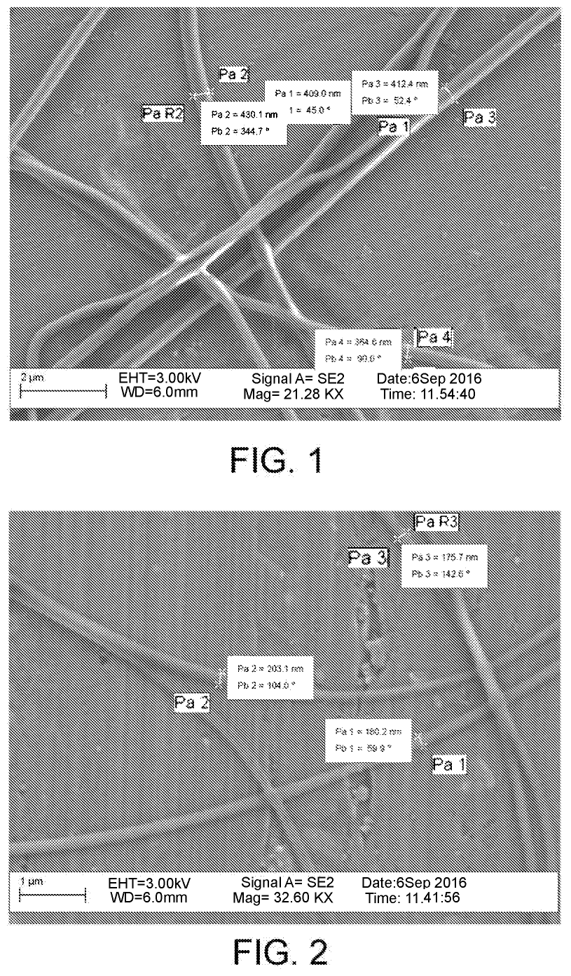

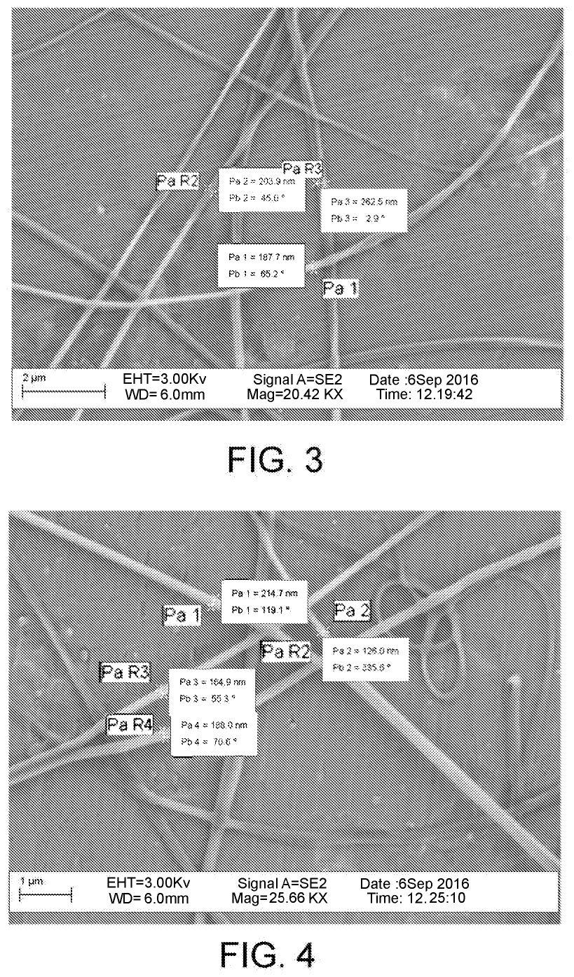

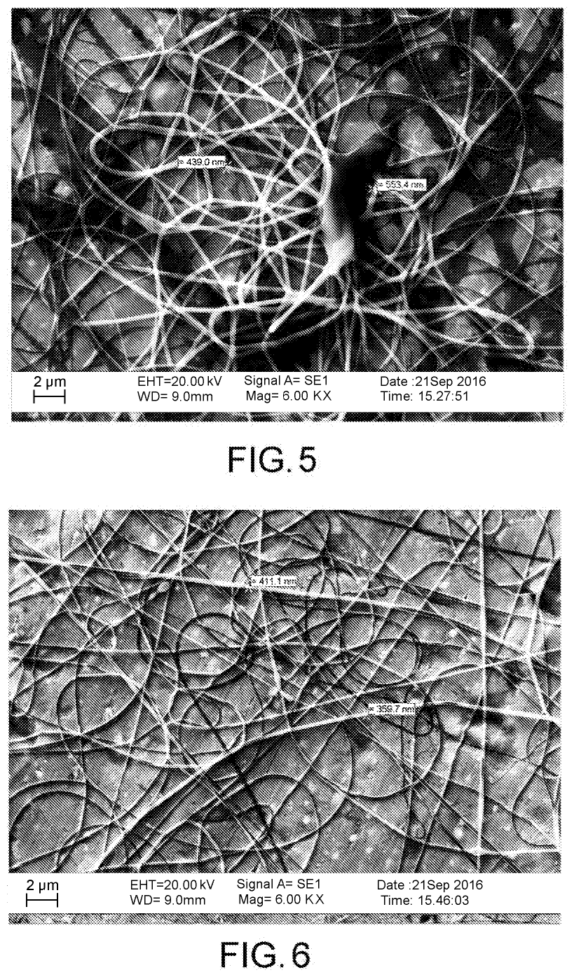

### DETAILED DESCRIPTION OF THE INVENTION

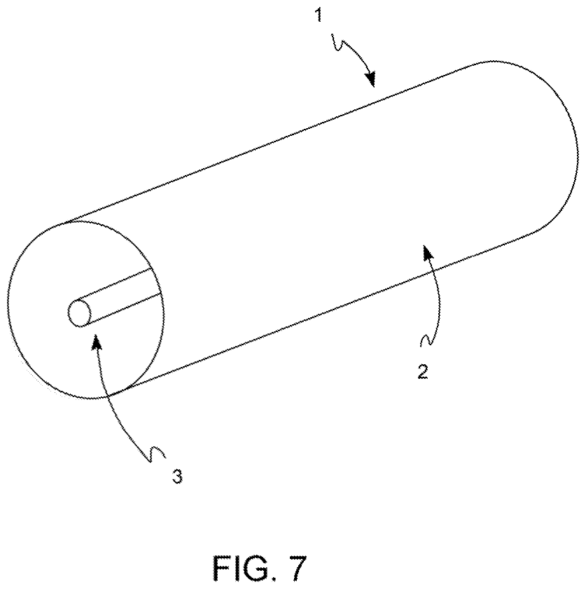

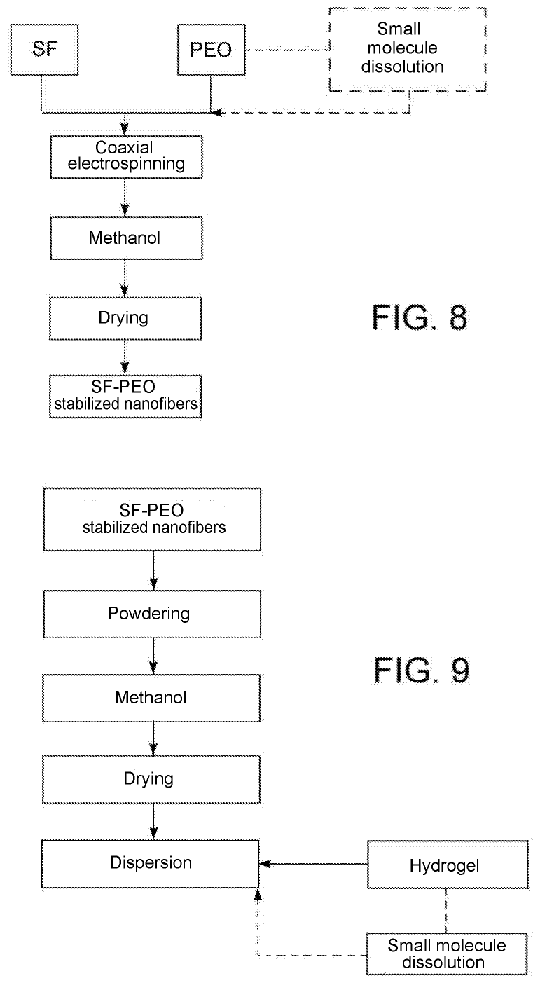

In a first aspect, with reference to FIG. 7, nanofibers (1) comprising an outer membrane (2) and a core (3), wherein said outer membrane (2) is made of fibroin and said core (3) is made of a biocompatible and biodegradable polymer and a method for obtaining thereof, are described hereinbelow.

Said fibroin is the natural or recombinant fibrous protein of silk. For example, it is obtained from domestic worm (genus Bombyx, mori species), from wild bug (genus Antheraea, species: pernyi, yamamai, militta, assama, etc., genus Philosamia, species cynthia ricini, etc.) from spider (various species of the Araneae order), from Hymenoptera, dipterans, etc., or by recombinant DNA technology known to those skilled in the art.

In a preferred embodiment, said polymer is selected from natural or synthetic biocompatible and biodegradable polymers, alone or in combination. Preferably, said polymer is water-soluble. By way of example, it is selected from the group comprising polyethylene oxide (PEO), polylactic acid (PLA), polyglycolic acid (PGA), PLA-PGA combinations or PLGA copolymers, polycaprolactone (PCL), hyaluronic acid, gelatin, collagen, chitosan, alginate, albumin. Even more preferably, said polymer is PEO.

Preferably, said nanofibers have a diameter from 50 to 2000 nm. In a preferred embodiment, said nanofibers have a diameter from 200 to 600 nm, preferably from 200 to 500 nm, where said diameter corresponds to the sum of the core thickness (3) and the outer membrane thickness (2).

Preferably, the thickness of said outer membrane is from 10 to 750 nm, or from 20 to 250 nm. By way of example, a nanofiber (1) according to the present invention has an outer membrane (2) with a thickness of 200 nm and a total diameter of 700 nm. Otherwise, it has an outer membrane (2) with a thickness of 100 nm and a total diameter of 300 nm, or an outer membrane (2) with a thickness of 100 nm and a total diameter of 400 nm, or an outer membrane (2) with a thickness of 60 nm and a total diameter of 250 nm.

Said core (3) extends longitudinally, preferably over the whole length of the nanofiber itself (1).

In a preferred embodiment, said nanofibers incorporate one or more bioactive molecules and/or particles and/or cells. Said bioactive molecules are, by way of example, selected from the group comprising anti-tumor, anti-coagulant, anti-thrombotic compounds, antibodies, vaccines, antibiotics, antivirals, anti-inflammatories, amino acids, peptides, proteins, enzymes, growth factors, angiogenic factors, nucleic acids, for example miRNA, salts, fibronectin, glycosaminoglycans, polysaccharides, vitamins, anti-oxidants, anti-microbials. By way of example, the anti-tumor drugs may comprise alkylating agents and the like (cyclophosphamide, ifosfamide, chlorambucil, melphalan, estramustine, lomustine, carmustine, carboplatin, cisplatin, oxaliplatin, busulfan, treosulfan, tiotepa, dacarbazine, procarbazine, temozolomide), antimetabolites (methotrexate, raltitrexed, pemetrexed, fluorouracil, capecitabine, cytarabine, gemcitabine, tegafur, fludarabine, cladribine, mercaptopurine, thioguanine, pentostatin, clofarabine, nelarabine), cytotoxic antibiotics (daunorubicin, doxorubicin, epirubicin, idarubicin, mitoxantrone, amsacrine, bleomycin, dactinomycin, mitomycin), natural origin derivatives (paclitaxel, docetaxel, vinblastine, vincristine, vindesine, vinorelbine, irinotecan, topotecan, trabectedin, etoposide, trabectedin), hormones and antagonists (diethylstilbestrol, ethinylestradiol, medroxyprogesterone, megestrol, norethisterone, gosereline, leuproreline, triptorelin, lanreotide, octreotide, tamoxifen, toremifene, fulvestrant, cyproterone, flutamide, bicalutamide, anastrozole, letrozole, exemestane), protein kinase inhibitors (dasatinib, erlotinib, imatinib, nilotinib, sunitinib, sorafenib), monoclonal antibodies (panitumumab, trastuzumab, rituximab, alemtuzumab, bevacizumab).

In a preferred embodiment, said one or more bioactive molecule is selected from the group comprising small water-soluble molecules, even more preferably, said small water-soluble molecules belong to the group of antibiotics with cytotoxic action. By way of example, Mitomycin C, Doxorubicin, Epirubicin, Gemcitabine are active ingredients particularly suitable for being loaded into the nanofibers according to the present invention.

Said particles are preferably submicrometric particles of inorganic type: quantum dots, magnetic nanoparticles, ceramics and metals (for example, platinum-, palladium-, rhodium-, gold-, silver-, copper-based); of organic type: liposomes, micelles, dendrimers, polymeric nanoparticles (PLA, PLGA, PVP, PEG, PCL, etc.) and nanogels (PAA, PVP, PVA, alginate, chitosan, collagen, fibrin, hyaluronic acid); organic-inorganic hybrids. In a preferred embodiment said particles are loaded with one or more bioactive molecules listed above and included in the polymer forming the core of the nanofibers of the present invention.

Said cells are stem cells, primary or line cells, in their native or engineered state. Preferably, said cells are stem cells, stromal cells, embryonic cells, endothelial cells, epithelial cells, muscle cells, fibroblasts, chondrocytes, osteoblasts, leukocytes, lymphocytes, or embryonic stem cells, for example iPSC. By way of example, said cells are engineered with a gene coding for a growth factor.

In a preferred embodiment, said nanofibers are obtained by an electrospinning method. Said nanofibers are characterized by being obtained by coaxial electrospinning and, by varying the concentration of the used solutions and the processing parameters, nanofibers with different diameters and with different ratios of core thickness (3) to outer membrane thickness (2) are obtained. Said coaxial electrospinning method comprises the following steps, not necessarily in the indicated sequence:

a) providing a fibroin solution (SF) at a concentration from 2 to 14% w/w in formic acid;
b) providing a water-soluble polymer solution in aqueous solution;
c) optionally, adding said bioactive molecules and/or cells in said aqueous solution;
d) filling at least two syringes, at least a first syringe with said fibroin solution and at least a second syringe with said water-soluble polymer solution;
e) connecting the syringes to the pumping system of a coaxial electrospinning system;
f) starting the electrospinning process.

In a preferred embodiment, said method comprises:

(a) providing a fibroin solution (SF) at a concentration from 2% to 14%, or from 5% to 12%, or from 6% to 12%, or from 5% to 10%, or from 7% to 10%, or from 7 to 9% w/w in formic acid, preferably in pure formic acid. Said solution with a high concentration of fibroin is generally obtained starting from aqueous fibroin solutions available on the market, typically at 5% w/w, from which a fibroin film is solidified, allowing the aqueous solution, poured over a suitable support, to dry under a hood. In a further embodiment, the aqueous fibroin solution is prepared starting from natural silk fibers deprived of sericin, applying a process known to those skilled in the art leading to the production of fibroin films. The film is then dissolved in formic acid, the dissolution is complete within 30-120 minutes, preferably 60-120 minutes, under stirring;
b) providing a water-soluble polymer solution in an aqueous solution, i.e. a saline or a culture medium or an enriched aqueous solution, or water, preferably bi-distilled water;
c) optionally, adding said bioactive molecules and/or particles and/or cells in said aqueous solution;
d) filling at least two syringes, at least a first syringe with said fibroin solution and at least a second syringe with said water-soluble polymer solution;
e) connecting the syringes to the pumping system of a coaxial electrospinning system; where said connection is carried out indifferently before or after said filling;
f) starting the electrospinning process by setting the parameters in the following ranges:
Flow rate: from 0.1 to 10 ml/h, preferably from 0.5 to 1 ml/h;
Working distance between spinneret (cathode) and metal collector (anode): from 5 to 100 cm, preferably from 10 to 80 cm;
Potential difference between cathode and anode: from 5 to 100 kV, preferably from 15 to 60 kV.

Particularly advantageous operating conditions have been demonstrated, providing at the cathode a voltage of −10 kV, at the anode of +10 kV, with a potential difference between cathode and anode of 20 kV, or at the cathode of −5 kV and at the anode of +15 kV, or at the cathode of −1 kV and at the anode of +19 kV, or at the cathode of 0 kV and at the anode of +24 kV.

In a preferred embodiment, said water-soluble polymer is PEO and said PEO solution has a concentration from 2 to 6%, preferably from 2.5% to 5% w/w in water, preferably bi-distilled water and is, for example, obtained by adding PEO powder to the water and leaving under stirring at room temperature, preferably for one night.

Preferably, said electrospinning process is carried out by working under a hood with activated suction, with a metal collector coated with a removable layer on which the electrospun fibers do not adhere such as for example an aluminum sheet, a polymeric membrane or a fabric, preferably with a static metal collector coated with an aluminum sheet.

In a further preferred embodiment, said electrospinning process is carried out with the following parameters:
Flow rate: from 1 to 0.5 ml/h;
Working distance between spinneret (cathode) and metal collector (anode): from 17 to 19 cm, preferably 18 cm;
Applied voltage: keeping the anode at 0 kV, the cathode is brought to 22-26 kV.

Preferably, said SF is at 10%, said PEO is at 5% and they are operated with a flow rate of 0.5 ml/h, at a distance of 18 cm with a voltage of 24 kV.

In a preferred embodiment, an E-Fiber® prototype coaxial electrospinning apparatus is used, provided by SKE Advanced Therapies in which the collector is coated with an aluminum sheet. Preferably, a stabilization chemical treatment follows said electrospinning process. Said stabilization treatment comprises soaking the product obtained from said process in an alcoholic bath, preferably a water and alcohol bath. For example, said alcohol is selected from methanol and ethanol, preferably ethanol, where said ethanol in said bath is from 50 to 100% v/v. Said soaking is prolonged for at least 10 minutes, or for a time of 20-minutes, preferably for a time of 20-40 minutes, or for 30 minutes. Subsequently, the thus stabilized product is withdrawn and removed from the aluminum sheet on which it was deposited and dried at room temperature.

The chemical stabilization treatment allows the nanofiber according to the present invention to preserve the nanofibrous structure, even when soaked in water, due to a crystallization process.

In said optional step c) of loading one or more active ingredients and/or particles and/or cells, the same are added to said aqueous solution, forming a suspension or a solution, depending on the type of the active ingredient, or the presence or absence of cells. Preferably, in this embodiment, the water-soluble polymer is added to said suspension or solution.

Experiments carried out using 0.1% Rhodamine B in water, with the sole purpose of monitoring the localization of the active ingredient during the preparation process, showed the typical Rhodamine staining in the obtained nanofiber core.

In a further aspect, the nanofibers according to the present invention are herein described in a powdered form. Said nanofibers are ground obtaining a powder with a particle size from 1 to 5000 microns, and preferably from 10 to 1000 microns. In an embodiment, said grinding is obtained manually, working in a mortar immersed in liquid nitrogen, thus obtaining a particle size from 100 to 1000 microns and a wide variability among the particle sizes, or operating with a mortar grinder, thus allowing to obtain smaller particle sizes with less variability. Alternatively, methods known to those skilled in the art such as mills, for example ball mills, or micronizers, such as circular-chamber air-jet, elliptical-chamber air-jet, opposed air-jet micronizers, are applied.

The thus obtained powder is collected and preferably recrystallized. Said recrystallization treatment comprises soaking the product obtained from said process in an alcoholic bath, preferably a water and alcohol bath. Said alcohol is selected, for example, from methanol and ethanol, preferably ethanol, where said ethanol in said bath is from 50 to 100% v/v. Said soaking is prolonged for at least 10 minutes, or for a time of 20-90 minutes, preferably for a time of 20-40 minutes, or for 30 minutes. The recrystallization takes place, for example, by collecting said powder in methanol and allowing said suspension to dry, preferably at room temperature in a container with a large surface. Subsequently, the dried product is repeatedly washed with water, preferably 2 or 3 washes are carried out in bi-distilled water, stirring and centrifuging, to eliminate any residual solvent. Sterilization is then carried out. Said sterilization process can be carried out using ethyl oxide, gamma-ray exposure, beta-ray exposure, UV-ray exposure; preferably UV-ray exposure.

When said nanofiber has been loaded with an active ingredient, which is a small hydrophilic molecule, it has been herein demonstrated that the latter remains in the ground nanofibers, as demonstrated by grinding nanofibers comprising the Rhodamine B dye, having it characteristics of a small hydrophilic molecule.

In a further aspect, a hydrogel system comprising the nanofibers according to the present invention and a method for the preparation of said system is herein described.

Said hydrogel is selected from the hydrogels known in the background art. In an embodiment, in which said system is developed for bladder applications, the hydrogel will preferably consist of hyaluronic acid and chondroitin sulfate. This is because the urothelium is coated with glycosaminoglycans and said coating, for example damaged for the removal of a tumor, can be favorably replaced by a hydrogel in hyaluronic acid and chondroitin sulfate, showing the latter a tropism for glycosaminoglycans. In addition, hyaluronic acid is adhesive for the CD44 molecules expressed by tumor cells and its presence in the bladder acts as a sequestering agent for any residual tumor cells after a tumor removal surgery.

The method for obtaining the hydrogel+nanofiber system according to the present invention comprises obtaining powdered nanofibers, optionally recrystallized and sterilized as described, and finally dispersing said powdered nanofibers into a solution, preferably sterile, of the chosen hydrogel.

In an embodiment, said nanofibers are dispersed in said hydrogel in a percentage from 0.01% to 5% w/v, or from 0.01% to 1% w/v, or from 0.1 to 0.8% w/v, or from 0.2 to 0.5% w/v. Concentrations higher than the preferred concentrations make the system too compact and difficult to be conveyed in a gel form.

In the embodiment comprising one or more active molecules and/or particles and/or cells in the hydrogel, the same are dissolved/dispersed in the desired amount in the hydrogel in which the powder is dispersed.

In a further embodiment, said powdered nanofibers are dispersed in an aqueous solution. Therefore, the present invention further relates to an aqueous solution comprising said powdered nanofibers.

The present invention further relates to said nanofibers, and/or said powdered nanofibers, and/or said powdered nanofibers dispersed in an aqueous solution, and/or said nanofibers powdered in a hydrogel, in which said nanofibers and/or said hydrogel are optionally loaded with one or more active molecules and/or particles and/or cells, for use to convey active ingredients and/or as a support for tissue regeneration.

For example, there are evidences supporting that the non-powdered nanofibers according to the present invention are advantageously used in the treatment for the containment of hernias and prolapses, reconstructive prostheses (e.g. mammary prostheses) and as scaffolds for the regeneration of tissues and organs of the peripheral nervous system (nerves), the vascular system (veins, arteries, arterio-venous fistulas for vascular accesses), the lymphatic system (lymphatic circulatory system, lymph nodes), the cardiovascular system (coronary arteries and cardiac muscle), the central nervous system (spinal cord), the skin and layers thereof, the tissues for containing and protecting internal organs (dura mater, pericardium, pleura, peritoneum, . . . ) and the musculoskeletal system tissues (tendons, ligaments, muscles, bones, cartilages, diaphragm), the respiratory system (nasal mucosa, trachea, larynx, pharynx, bronchi, lungs), the digestive system (esophagus, stomach, intestine, tissues of the anal canal), the oral cavity tissues and mucous membranes (gums, teeth, the tongue, the urinary system (kidneys, ureters, bladder, urethra, adrenal glands), the genital and reproductive system (corpora cavernosa, prostate, uterus, vulva, vagina, endometrium, fallopian tubes).

Likewise, said powdered and/or dispersed in an aqueous solution and/or incorporated in hydrogels nanofibers can be included in systems and/or scaffolds for the repair and regeneration of all the tissues listed in the previous paragraph.

The nanofiber-based systems according to the present invention (not powdered, powdered, dispersed in an aqueous solution, dispersed in hydrogel) can be used for the regeneration of tissues and/or organs following damage thereof due to pathologies, traumas or surgical procedures as, for example, in the case of total or partial resection following the removal of tumors.

In a preferred embodiment, in which said hydrogel is hyaluronic acid+chondroitin sulfate and said active ingredient is Mitomycin C, said system is advantageously applied in the treatment of bladder cancer, in particular in the tumor post-removal therapy.

In an embodiment, the present invention relates to nanofibers (1) comprising an outer membrane (2) and a core (3) wherein said outer membrane (2) is made of fibroin and said core (3) is made of polyethylene oxide (PEO).

Preferably, said nanofibers have a diameter from 200 to 500 nm. Preferably, the core of said nanofibers comprises one or more hydrophilic molecules.

In an embodiment, the present invention further relates to a method for obtaining nanofibers wherein said method comprises:
providing a fibroin solution (SF) at a concentration from 6 to 12%, preferably from 8% to 10% w/w in pure formic acid;
providing a PEO solution at a concentration from 2 to 6%, preferably from 2.5% to 5% w/w in water, preferably bi-distilled water;
optionally, dissolving one or more small hydrophilic molecules in said water;
filling two syringes with said solutions and connecting them to the pumping system of a coaxial electrospinning system;
starting the electrospinning process by setting the parameters in the following ranges: Flow rate: from 1 to 0.5 ml/h, Working distance between spinneret (cathode) and metal collector (anode): from 17 to 19 cm, preferably 18 cm, Applied voltage: keeping the anode at 0 kV, the cathode is brought to 22-26 kV.

Preferably, said method also comprises a stabilization step in which the product obtained from the described process is soaked into methanol for about 30′.

In a further embodiment, present invention relates to a hydrogel system comprising nanofibers according to the present invention, characterized in that said nanofibers are powdered and have a particle size from 10 to 1000 microns. Preferably, said hydrogel comprises hyaluronic acid+chondroitin sulfate. Preferably, said nanofibers and/or said hydrogel comprise an active ingredient which is preferably selected from the group comprising: Mitomycin C, Doxorubicin, Epirubicin, Gemcitabine.

In a further embodiment, the present invention relates to a method for obtaining the described system comprising:
providing nanofibers according to the present invention;
grinding said nanofibers obtaining a powder with a particle size from 10 to 1000 microns, preferably from 100 to 1000 microns;
optionally, recrystallizing in methanol;
dispersing in the hydrogel, optionally added with one or more hydrophilic small molecules.

In a further embodiment, the present invention relates to said system for the use as a carrier for active ingredients and/or as a support for tissue regeneration. Preferably, said system comprises, as an active ingredient, Mitomycin C for use in the treatment of bladder cancer.

The advantage when using fibroin compared to other biopolymers, such as chitosan, is apparent since fibroin, although being biodegradable, has a greater resistance to the aqueous environment.

Furthermore, fibroin has amazing biomimetic capabilities. This allows the nanofiber system according to the present invention to be not only a carrier for active ingredients but also to act per se as a support for tissue regeneration, since the nanofibers according to the present invention are able to be integrated into the system in which they are placed.

A further advantage is the surprising greater mechanical resistance to the grinding of fibroin compared to other biopolymers. For example, it would not have been possible to obtain the powdering according to the present invention with the chitosan-PEO nanofibers known in the background art, since the powdering process destroys the nanofiber structure of the chitosan-PEO nanofibers known in the background art. Only with the nanofibers according to the present invention it is therefore possible to obtain a powder preserving the nanofiber structure. Moreover, fibroin has a programmable biodegradability, i.e., by varying the thickness of the outer membrane of the nanofiber, the biodegradability of fibroin can be modulated and thus the release of the active ingredient dissolved in the core of the nanofiber itself can be controlled. These characteristics allow an amazing control of the in vivo release kinetics, making it possible to finely modulate fibroin biodegradation for prolonged periods and, consequently, the release of the active ingredient conveyed by the system according to the present invention.

It has been observed that advantageously the greater dimensional variability obtained with the manual grinding of the nanofibers brings a release of the active ingredient contained in the nanofibers themselves by the continuous system over time, since the release is faster from smaller fragments and delayed from fragments of larger dimensions. Having a large size distribution of the fragments allows therefore to better distribute over time the release of the active ingredient.

### EXAMPLES

### Example 1: Characterization of the Nanofibers Obtained by Varying the Electrospinning Parameters and the Concentrations of the Starting Solutions

Table 1 below shows the applied parameters and the obtained results. The last column in the table refers to the representative figures of nanofibers obtained according to the described procedure.

TABLE 1

| | | | | | | Thickness | |
|---|---|---|---|---|---|---|---|
| | | | | Working | | of | |
| | SF | PEO | Flow speed | distance | Voltage | nanofibers | |
| Combinations | [%] | [%] | (ml/h) | (cm) | (kV) | (nm) | FIG. |
| 1 | 8 | 5 | 1 | 18 | 22 | ≈400 | 1 |
| 2 | 8 | 2.5 | 1 | 18 | 22 | ≈200 | 2 |
| 3 | 8 | 2.5 | 1 | 18 | 24 | ≈200 | 3 |
| 4 | 8 | 2.5 | 1 | 18 | 26 | ≈200 | 4 |
| 5 | 10 | 5 | 1 | 18 | 22 | ≈500 | 5 |
| 6 | 10 | 5 | 0.5 | 18 | 24 | ≈400 | 6 |

For the purposes of the present invention, the combination number 6 has proved particularly advantageous. In fact, it has been found that with said process the electrospinning process is more stable and highly reproducible.

### Example 2: Non-Toxicity Test of the Hydrogel+Nanofiber System

A hepatocarcinoma cell line (HepG2) was treated with hydrogel alone, or with the hydrogel+nanofiber system according to combination 6 of Example 1, where the hydrogel included hyaluronic acid and chondroitin sulfate.

Cells were observed, and their viability was quantized with the MTT assay after 24 and 48 h from the treatment.

A cellular viability of 124% and 82% at 24 and 48 h was observed, respectively, exposing cells to the hydrogel+nanofiber system, where the % is expressed with respect to 100% measured in control cells, i.e. cells not exposed to the system hydrogel+nanofibers. In the presence of the hydrogel alone, a cell viability of 119% and 126% was observed at 24 and 48 h, respectively. The data show that the system according to the present invention is not toxic.

What is claimed is:

1. Nanofibers comprising an outer membrane and a core, wherein said outer membrane is made of fibroin and said core is a biocompatible and biodegradable polymer.

2. The nanofibers according to claim 1, wherein said fibroin is the natural or recombinant protein of silk.

3. The nanofibers according to claim 1, wherein said biocompatible and biodegradable polymer is a natural or synthetic polymer, used alone or in combination.

4. The nanofibers according to claim 1, wherein said biocompatible and biodegradable polymer is a water-soluble polymer.

5. The nanofibers according to claim 1, wherein said biocompatible and biodegradable polymer is one or more polymers selected from the group comprising polyethylene oxide (PEO), polycaprolactone (PCL), hyaluronic acid, gelatin, collagen, chitosan, alginate, albumin.

6. The nanofibers according to claim 1, wherein said biocompatible and biodegradable polymer is PEO.

7. The nanofibers according to claim 1, wherein said nanofibers have a diameter from 50 to 2000 nm.

8. The nanofibers according to claim 7, wherein said nanofibers have a diameter from 200 to 500 nm.

9. The nanofibers according to claim 1, wherein a thickness of said outer membrane is from 10 to 750 nm, or from 20 to 250 nm.

10. The nanofibers according to claim 1, wherein said outer membrane has a thickness of 200 nm and said nanofiber has a diameter of 700 nm, or an outer membrane (2) with a thickness of 100 nm and a diameter of 300 nm, an outer membrane (2) with a thickness of 100 nm and a diameter of 400 nm, or an outer membrane (2) with a thickness of 60 nm and a diameter of 250 nm.

11. The nanofibers according to claim 1, wherein said core extends longitudinally.

12. The nanofibers according to claim 1, wherein said core comprises one or more bioactive molecules and/or particles and/or cells.

13. The nanofibers according to claim 12, wherein said bioactive molecules are selected from the group comprising anti-tumor, anti-coagulant, anti-thrombotic compounds, antibodies, vaccines, antibiotics, antivirals, anti-inflammatories, amino acids, peptides, proteins, enzymes, growth factors, angiogenic factors, nucleic acids, salts, fibronectin, glycosaminoglycans, polysaccharides, vitamins, anti-oxidants, anti-microbials.

14. The nanofibers according to claim 12, wherein said bioactive molecules are selected from the group comprising anti-tumor compounds including alkylating agents comprising cyclophosphamide, ifosfamide, chlorambucil, melphalan, estramustine, lomustine, carmustine, carboplatin, cisplatin, oxaliplatin, busulfan, treosulfan, tiotepa, dacarbazine, procarbazine, temozolomide; antimetabolites comprising methotrexate, raltitrexed, pemetrexed, fluorouracil, capecitabine, cytarabine, gemcitabine, tegafur, fludarabine, cladribine, mercaptopurine, thioguanine, pentostatin, clofarabine, nelarabine; cytotoxic antibiotics comprising daunorubicin, doxorubicin, epirubicin, idarubicin, mitoxantrone, amsacrine, bleomycin, dactinomycin, mitomycin; derivatives of natural origin comprising paclitaxel, docetaxel, vinblastine, vincristine, vindesine, vinorelbine, irinotecan, topotecan, trabectedin, etoposide, trabectedin; hormones and antagonists comprising diethylstilbestrol, ethinylestradiol, medroxyprogesterone, megestrol, norethisterone, gosereline, leuprorelin, triptorelin, lanreotide, octreotide, tamoxifen, toremifene, fulvestrant, cyproterone, flutamide, bicalutamide, anastrozole, letrozole, exemestane; protein kinase inhibitors comprising dasatinib, erlotinib, imatinib, nilotinib, sunitinib, sorafenib; and monoclonal antibodies comprising panitumumab, trastuzumab, rituximab, alemtuzumab, bevacizumab.

15. The nanofibers according to claim 12, wherein said bioactive molecules are selected from the group comprising small hydrophilic molecules.

16. The nanofibers according to claim 15, wherein said small molecules belong to the group of antibiotics with cytotoxic action including Mitomycin C, Doxorubicin, Epirubicin, and Gemcitabine.

17. The nanofibers according to claim 12, wherein said particles are of inorganic type, organic type, and organic-inorganic hybrids.

18. The nanofibers according to claim 12, wherein said cells are stem cells, primary or line cells, in the native or engineered state.

19. A method for obtaining nanofibers according to claim 1, wherein said method comprises the following steps, not necessarily in the indicated sequence:
a) providing a fibroin solution at a concentration from 2 to 14% w/w in formic acid;
b) providing a water-soluble polymer solution in an aqueous solution;
c) optionally, adding said bioactive molecules and/or particles and/or cells in said aqueous solution;
d) filling at least two syringes, at least a first syringe with said fibroin solution and at least a second syringe with said water-soluble polymer solution;
e) connecting the syringes to a pumping system of a coaxial electrospinning system; and
f) starting an electrospinning process.

20. The method according to claim 19, wherein said fibroin solution is at a concentration from 2 to 14%, or from 5 to 12%, or from 6 to 12%, or from 5 to 10%, or from 7% to 10%, or from 7 to 9% w/w in formic acid.

21. The method according to claim 19, wherein said aqueous solution is a saline or a culture medium, an enriched aqueous solution, or water.

22. The method according to claim 19, wherein said electrospinning process is performed by setting parameters in the following ranges:
Flow rate: from 0.1 to 10 ml/h;
Working distance between spinneret (cathode) and metal collector (anode): from 5 to 100 cm;
Potential difference between said cathode and said anode: from 5 to 100 kV.

23. The method according to claim 22, wherein said cathode has a voltage of −10 kV and said anode of +10 kV, or said cathode −5 kV and said anode +15 kV, or said cathode −1 kV and said anode +19 kV, or said cathode 0 kV and said anode +24 kV.

24. The method according to claim 19, wherein said electrospinning process is carried out by working under a hood with activated suction, with a metal collector coated with a removable layer on which the electrospun fibers do not adhere, including an aluminum sheet, a polymeric membrane, or a fabric.

25. The method according to claim 19 comprising:
providing said fibroin solution at a concentration from 6 to 12% w/w in pure formic acid;
providing said water-soluble polymer solution as a PEO solution at a concentration from 2 to 6% w/w in water;
optionally, dissolving one or more small hydrophilic molecules in said water;
filling two syringes with said solutions and connecting them to the pumping system of the coaxial electrospinning system;
starting the electrospinning process by setting the parameters in the following ranges: Flow rate: from 1 to 0.5 ml/h, Working distance between spinneret (cathode) and metal collector (anode): from 17 to 19 cm, Applied voltage: keeping the anode at 0 kV, the cathode is brought to 22-26 kV.

26. The method according to claim 19, further comprising a product stabilization step.

27. The method according to claim 26, wherein said product stabilization step is a chemical treatment comprising soaking the product obtained by electrospinning in an alcohol and/or water bath for at least 10 minutes.

28. The method according to claim 27, wherein in said product stabilization step, said alcohol is selected from methanol and ethanol, wherein in said bath, said ethanol is from 50 to 100% v/v.

29. The method according to claim 27, wherein in said product stabilization step, said soaking is prolonged for a time of 20-90 minutes.

30. The method according to claim 27, wherein said product is soaked in methanol for about 30 minutes.

31. Nanofibers according to claim 1, which are powdered and have a particle size from 1 to 5000 microns.

32. An aqueous solution comprising powdered nanofibers according to claim 31.

33. A system that is a hydrogel comprising powdered nanofibers according to claim 31.

34. The system according to claim 33, wherein said powdered nanofibers are in a concentration from 0.01% to 5% w/v, or from 0.01% to 1% w/v, or from 0.1 to 0.8% w/v, or from 0.2 to 0.5% w/v.

35. The system according to claim 33, wherein said hydrogel comprises hyaluronic acid+chondroitin sulfate.

36. The system according to claim 33, wherein said hydrogel comprises one or more bioactive molecules and/or particles and/or cells.

37. The system according to claim 36, wherein said one or more bioactive molecules are selected from the group comprising anti-tumor, anti-coagulant, anti-thrombotic compounds, antibodies, vaccines, antibiotics, antivirals, anti-inflammatories, amino acids, peptides, proteins, enzymes, growth factors, angiogenic factors, nucleic acids, salts, fibronectin, glycosaminoglycans, polysaccharides, vitamins, anti-oxidants, and anti-microbials.

38. The system according to claim 36, wherein said nanofibers and/or said hydrogel comprise an active ingredient selected from the group comprising: Mitomycin C, Doxorubicin, Epirubicin, and Gemcitabine.

39. A method for obtaining a system according to claim 33, said method comprising:
providing nanofibers comprising an outer membrane and a core wherein said outer membrane is made of fibroin and said core is a biocompatible and biodegradable polymer;
grinding said nanofibers to obtain a powder having a particle size from 1 to 5000 microns;
optionally, re-crystallizing; and
dispersing in the hydrogel, optionally added with one or more bioactive molecules and/or particles and/or cells.

40. A method of regenerating tissues in humans or animals, said method comprising administering the nanofibers according to claim 1 or a hydrogel containing said nanofibers.

41. The method according to claim 40, wherein said nanofibers and/or said hydrogel are carriers for bioactive molecules and/or particles and/or cells.

42. (canceled)

43. A method of treating humans or animals, said method comprising administering the nanofibers according to claim 1 or a hydrogel containing said nanofibers for the containment of hernias and prolapses, reconstructive prostheses; or as a scaffold for the regeneration of tissues and organs of the peripheral nervous system, the vascular system, including veins, arteries, arterio-venous fistulas for vascular accesses, the lymphatic system, including lymphatic circulatory system, lymph nodes, the cardiovascular system, including coronary arteries and cardiac muscle, the central nervous system, including spinal cord, the skin and layers thereof, the tissues for containing and protecting internal organs, including dura mater, pericardium, pleura, peritoneum, and the tissues of the muscular-skeletal system, including tendons, ligaments, muscles, bones, cartilages, diaphragm, the respiratory system, including nasal mucosa, trachea, larynx, pharynx, bronchi, lungs, the digestive system, including esophagus, stomach, intestines, tissues of the anal canal, tissues and mucous membranes of the oral cavity, including gums, teeth, tongue, the urinary system, including kidneys, ureters, bladder, urethra, adrenal glands, or the genital and reproductive system, including corpora cavernosa, prostate, uterus, vulva, vagina, endometrium, fallopian tubes.

44. A method of treating bladder cancer, said method comprising administering the nanofibers according to claim 1 or a hydrogel containing said nanofibers.

45. The method according to claim 40, wherein said nanofibers are in a hydrogel system and said system comprises Mitomycin C as an active ingredient.
